(19) United States
(12) Patent Application Publication
(10) Pub. No.: US 2025/0295831 A1
(43) Pub. Date: Sep. 25, 2025

(54) ARTIFICIAL BLOOD VESSEL AND METHOD OF MANUFACTURING ARTIFICIAL BLOOD VESSEL

(71) Applicant: HI-LEX CORPORATION, Hyogo (JP)

(72) Inventor: Shinsaku KOARASHI, Hyogo (JP)

(21) Appl. No.: 19/085,086

(22) Filed: Mar. 20, 2025

(30) Foreign Application Priority Data
Mar. 22, 2024 (JP) 2024-046232

(51) Int. Cl. A61L 27/16; A61F 2/06; A61L 27/50

(52) U.S. Cl. CPC A61L 27/16; A61F 2/06; A61L 27/507; A61F 2230/0091; A61F 2240/001; A61F 2250/0015

(57) ABSTRACT

It is an object of the present invention to provide a highly flexible artificial blood vessel and a method of manufacturing the artificial blood vessel. The artificial blood vessel VE of the present invention is an artificial blood vessel composed of ePTFE having nodes and fibrils formed between the nodes, wherein high-density regions R1 and low-density regions R2 are alternately provided in an axial direction D1 of the artificial blood vessel VE, in the high-density regions R1, the nodes and the fibrils are in a compressed and densely packed state in the axial direction D1, and in the low-density regions R2, the nodes and the fibrils are in a lower density state compared to the high-density region R1.

### CROSS-REFERENCE TO RELATED APPLICATIONS

This application claims priority to JP Application No. 2024-46232, filed Mar. 22, 2024, the disclosure of which is expressly incorporated by reference herein in its entirety.

### TECHNICAL FIELD

The present invention relates to an artificial blood vessel and a method of manufacturing the artificial blood vessel.

### BACKGROUND OF THE INVENTION

An artificial blood vessel made of expanded polytetrafluoroethylene (hereinafter referred to as ePTFE) has been used as a material of an artificial blood vessel. The artificial blood vessel made of ePTFE has a structure having nodes and fibrils formed between the nodes, as shown in JP 2005-530549 A, by forming polytetrafluoroethylene (PTFE) into a tubular shape and rapidly elongating it.

### SUMMARY OF THE INVENTION

The artificial blood vessel made of ePTFE is biocompatible and flexible, but there has been a demand for an artificial blood vessel made of ePTFE that is further flexible.

Therefore, it is an object of the present invention to provide a highly flexible artificial blood vessel and a method of manufacturing the artificial blood vessel.

The artificial blood vessel of the present invention is an artificial blood vessel composed of ePTFE having nodes and fibrils formed between the nodes, wherein high-density regions and low-density regions are alternately provided in an axial direction of the artificial blood vessel, wherein, in the high-density regions, the nodes and the fibrils are in a compressed and densely packed state in the axial direction, and in the low-density regions, the nodes and the fibrils are in a lower density state compared to the high-density region.

Moreover, the method of manufacturing the artificial blood vessel of the present invention comprises the steps of: a) providing a tubular artificial blood vessel base material composed of ePTFE having nodes and fibrils formed between the nodes; b) compressing the artificial blood vessel base material in an axial direction of the artificial blood vessel base material in a state in which a core member is inserted inside the artificial blood vessel base material; c) releasing a force compressing the artificial blood vessel base material to extend the artificial blood vessel base material; d) re-compressing the extended artificial blood vessel base material one or more times; and e) re-extending the artificial blood vessel base material compressed in the step d) core member.

According to the artificial blood vessel and the method of manufacturing the artificial blood vessel of the present invention, a high flexible artificial blood vessel can be provided.

### BRIEF DESCRIPTION OF THE DRAWINGS

FIG. 1 is a schematic view of an artificial blood vessel according to one embodiment of the present invention.

FIG. 2 is an enlarged schematic view of an area A1 of FIG. 1.

FIG. 3 is a 25× SEM photograph of a surface of a sample piece obtained by cutting a part of an artificial blood vessel, as taken in a wall thickness direction.

FIG. 4 is a 100× SEM photograph of an area A2 of FIG. 3.

FIG. 5 is a view showing a preparation step of an artificial blood vessel base material in the method of manufacturing an artificial blood vessel.

FIG. 6 is a view showing a first baking step in the method of manufacturing an artificial blood vessel.

FIG. 7 is a view showing a marker application step in the method of manufacturing an artificial blood vessel.

FIG. 8 is a view showing a first compression step in the method of manufacturing an artificial blood vessel.

FIG. 9 is a view showing a belt-shaped portion forming step in the method of manufacturing an artificial blood vessel.

FIG. 10 is a view showing a first extension step in the manufacturing method for an artificial blood vessel.

FIG. 11 is a view showing an additional compression step in the method of manufacturing an artificial blood vessel.

FIG. 12 is a view showing an additional extension step in the method of manufacturing an artificial blood vessel.

FIG. 13 is a 33× SEM photograph of a sample piece obtained by cutting a part of an artificial blood vessel, as taken obliquely.

FIG. 14 is a 130× SEM photograph showing a cut surface of a sample piece obtained by cutting a part of an artificial blood vessel, as taken in an axial direction.

FIG. 15 is a photograph showing a belt-shaped portion of an artificial blood vessel.

FIG. 16 is a schematic view showing a method of evaluating flexibility of an artificial blood vessel.

FIG. 17 is a microscopic image of a sample piece obtained by cutting a part of an artificial blood vessel, as taken with a microscope in a wall thickness direction.

### DETAILED DESCRIPTION

An artificial blood vessel and a method of manufacturing the artificial blood vessel according to one embodiment of the present invention will be described below with reference to the drawings. Besides, embodiments shown below are merely examples, and the artificial blood vessel and the method of manufacturing the artificial blood vessel of the present invention are not limited to the following embodiments.

Besides, in the present specification, “perpendicular to A” and similar expressions do not only refer to a direction strictly perpendicular to A, but also refer to the direction including being substantially perpendicular to A. Moreover, in the present specification, “parallel to B” and similar expressions do not only refer to a direction strictly parallel to B, but also refer to the direction including being substantially parallel to B. In addition, in the present specification, “C-shape” and similar expressions do not only refer to a strict C-shape, but also refer to the shape including a shape visually associated with a C-shape (substantially a C-shape).

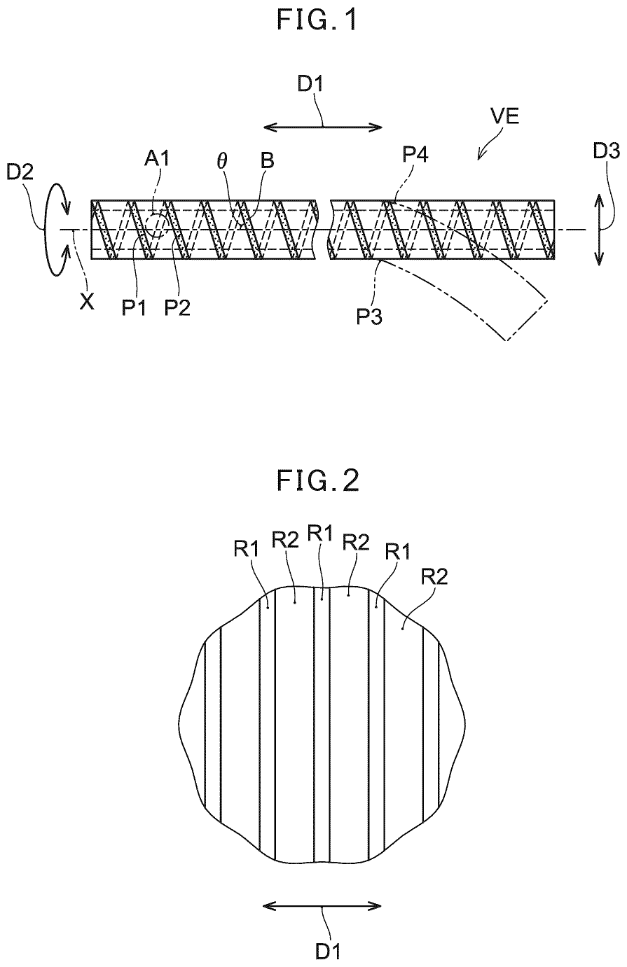

The artificial blood vessel VE (see FIG. 1) according to one embodiment of the present invention is used, such as, for example, for replacing a pathological living blood vessel and bypassing the living blood vessel. The artificial blood vessel VE is composed of a tubular body having a predetermined length.

A diameter of the artificial blood vessel VE can be changed depending on a site to be used, etc., and is not particularly limited. For example, the artificial blood vessel VE may be an artificial blood vessel with a large diameter having an inner diameter of 10 mm or more (for a thoracoabdominal aorta), an artificial blood vessel with a medium diameter having an inner diameter of 6 mm or more and less than 10 mm, such as 6 mm or 8 mm (for lower limb arteries, carotid artery and axillary arteries), or an artificial blood vessel with a small diameter having an inner diameter of less than 6 mm, such as 4 mm or 5 mm. A wall thickness of the artificial blood vessel VE is appropriately changed depending on an inner diameter and a length of the artificial blood vessel to be used and is not particularly limited. For example, the wall thickness of the artificial blood vessel VE can be 0.1 to 2 mm. For example, when the inner diameter of the artificial blood vessel VE is 5 to 6 mm, the wall thickness can be 0.3 to 0.7 mm, preferably 0.4 to 0.6 mm.

A length of the artificial blood vessel VE in an axial direction D1 can be changed depending on a site to be used and is not particularly limited. For example, the length of the artificial blood vessel VE in the axial direction D1 can be 50 to 1000 mm.

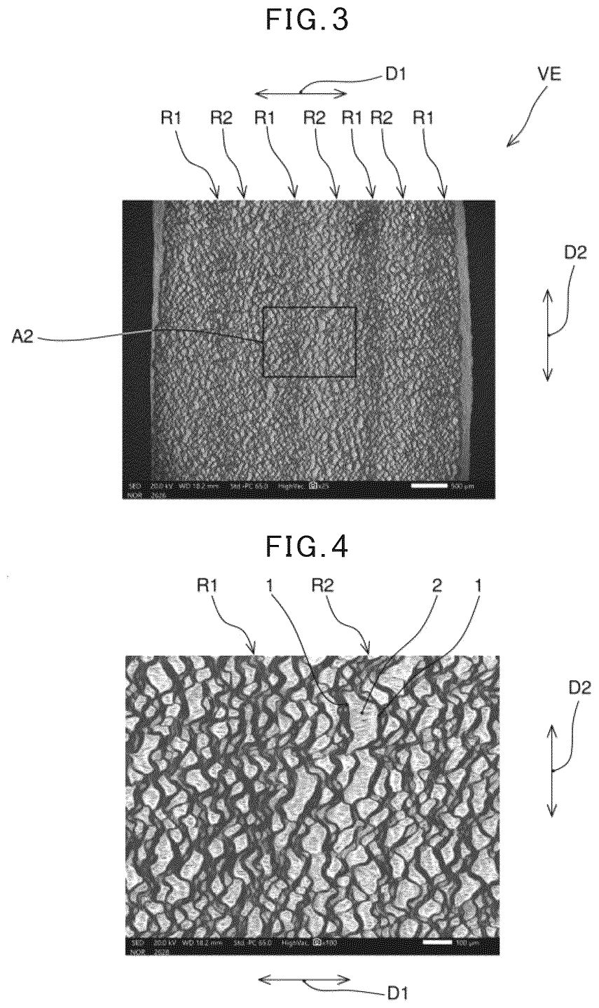

The artificial blood vessel VE of the present embodiment is composed of ePTFE. Specifically, the artificial blood vessel VE is composed of ePTFE having nodes 1 and fibrils 2 formed between the nodes 1, as shown in FIG. 4. FIG. 3 is a 25× SEM photograph taken in a wall thickness direction from the outer surface of the artificial blood vessel VE as obtained by cutting a tubular artificial blood vessel VE manufactured by the manufacturing method mentioned below with a cutter knife into a sample piece, flattening the sample piece, and then subjecting it to Au sputter coating. FIG. 4 is an enlarged photograph of the area A2 in FIG. 3.

For the artificial blood vessel made of ePTFE, at first, a lubricant is mixed with an unsintered PTFE powder to prepare a mixture, as shown in, for example, Japanese Examined Patent Application Publication No. S42-13560. This mixture is extruded into a tubular shape using a ram extruder, and the tube is then elongated in an axial direction at a desired elongating ratio. While the obtained tube is fixed to prevent shrinkage and is heated to a sintering temperature or higher, the elongated structure is sintered and fixed. Accordingly, a tubular artificial blood vessel base material made of ePTFE is obtained. By subjecting a predetermined treatment mentioned below, an artificial blood vessel VE is obtained. It should be noted that a method of manufacturing an artificial blood vessel made of ePTFE (artificial blood vessel base material) is not limited to the above-described method, as long as it is a method by which a structure having nodes and fibrils can be obtained.

A porosity and a fibril length of the artificial blood vessel base material can be set arbitrarily by adjusting an elongating ratio and a rate of strain in elongating. A tube that is a base of the artificial blood vessel base material is elongated in one axial direction. The elongating ratio is not particularly limited, but is selected within a range of, for example, 1.2 to 15 times, preferably 2 to 10 times, and more preferably 2 to 5 times. The sintering temperature for sintering the artificial blood vessel base material is not particularly limited, but can be, for example, 350 to 800° C.

The artificial blood vessel base material is manufactured, in an extrusion molding step, at an extrusion molding speed calculated as a product of an extrusion reduction ratio (hereinafter may be referred to as an “extrusion RR”) and a ram speed (mm/min), which is used for manufacturing a publicly-known artificial blood vessel base material.

In order to improve extrusion moldability at a high speed, it is considered that it is preferable to set a compounding ratio of a liquid lubricant to an unsintered PTFE powder to a relatively higher ratio, but compounding an excessive amount of liquid lubricant may result in a decrease in strength of the artificial blood vessel base material. Therefore, it is desirable to set the compounding ratio of the liquid lubricant to preferably 30 parts by mass or less, and more preferably 26 parts by mass or less, based on 100 parts by mass of the unsintered PTFE powder. A lower limit of the compounding ratio of the liquid lubricant is preferably 15 parts by mass, more preferably 18 parts by mass, and particularly preferably 20 parts by mass, based on 100 parts by mass of the unsintered PTFE powder. A compounding amount of the liquid lubricant based on 1 kg of unsintered PTFE powder is desirably kept to preferably 380 ml or less, and more preferably 330 ml or less.

The nodes 1 are connected three-dimensionally in the artificial blood vessel VE (see FIGS. 3, 4, 13, and 14). Specifically, the nodes 1 are connected to each other in an axial direction D1, a circumferential direction D2, and a radial direction D3 (see FIG. 1) of the artificial blood vessel VE. Moreover, as shown in FIG. 4, the fibrils 2 extend in the axial direction D1 so as to connect between a part of the node 1 and another part of the node 1 that are spaced apart in the axial direction D1.

In the present embodiment, as shown in FIGS. 2 and 3, high-density regions R1 and low-density regions R2 are alternately provided in the axial direction D1 of the artificial blood vessel VE. In the high-density regions R1, the nodes 1 and the fibrils 2 are in a compressed and densely packed state in the axial direction D1, and in the low-density regions R2, the nodes 1 and the fibrils 2 are in a lower density state compared to the high-density region R1.

The high-density region R1 is a region where the nodes 1 and the fibrils 2 (particularly the nodes 1) have a relatively higher density compared to the low-density region R2. The high-density region R1 is a compressed site where the nodes 1 and the fibrils 2 are in a compressed and densely packed state in the axial direction D1 of the artificial blood vessel VE. As shown in FIGS. 2 and 3, the high-density regions R1 are provided annularly in the circumferential direction D2 of the artificial blood vessel VE. A structure and a forming method of the high-density region R1 are not particularly limited as long as the high-density region R1 is configured so that the nodes 1 and the fibrils 2 are densely packed in a compressed state compared to the other region (low-density region R2). In the present embodiment, the high-density region R1 is composed of a compressed stripe portion that is produced when a tubular artificial blood vessel base material VEB is compressed in the axial direction D1 (see FIG. 8) (a portion having a folding crease centered at the bottom of the valley of the artificial blood vessel base material VEB which has shrunk into a bellows shape upon compression of the artificial blood vessel base material VEB in the axial direction D1). In the present embodiment, the high-density region R1 provided as the compressed stripe is compressed in the axial direction D1 with a core member C (see FIG. 8, etc.) inserted into the artificial blood vessel base material VEB, as mentioned below. In this case, parts displaced outward in the radial direction D3 and parts displaced inward in the radial direction D3, of the artificial blood vessel base material VEB, are created, and the parts displaced inward in the radial direction D3 come into contact with the core member C to facilitate becoming a high density. It should be noted that respective widths of the high-density region R1 and the low-density region R2 in the axial direction D1 and a ratio of their widths to each other can be appropriately changed depending on a method of compressing an artificial blood vessel base material or an artificial blood vessel base material used, and are not particularly limited. In addition, a boundary between the high-density region R1 and the low-density region R2 is not clearly defined as shown in FIG. 3, but it only needs to be confirmed, when the surface of the artificial blood vessel VE is enlarged, for example in an SEM photograph, that there are alternating high-density regions R1 that can be understood to have a high density (regions in a dark color when viewed) and low-density regions R2 that can be understood to have a lower density than the high-density regions R1 (regions in a light color when viewed). Alternatively, in an image obtained by capturing the high-density region R1 and the low-density region R2, a boundary between the high-density region R1 and the low-density region R2 may be determined with a brightness contrast or the like. It should be noted that specific numerical values of density of the nodes 1 and the fibrils 2 in the high-density region R1 are not limited as long as they are relatively higher compared to density of the nodes 1 and the fibrils 2 in the low-density region R2.

The low-density region R2 is a region where the nodes 1 and the fibrils 2 (particularly the nodes 1) have a relatively lower density compared to the high-density region R1. The low-density region R2 is a non-compressed site sandwiched in the axial direction D1 by high-density regions R1 where the nodes 1 and the fibrils 2 are in the compressed and densely packed state in the axial direction D1 of the artificial blood vessel VE. As shown in FIGS. 2 and 3, the low-density regions R2 are provided annularly in the circumferential direction D2 of the artificial blood vessel VE. A structure and a forming method of the low-density region R2 are not particularly limited as long as the low-density region R2 is configured so that the nodes 1 and the fibrils 2 have a lower density compared to the other region (high-density region R1). In the present embodiment, the low-density region R2 is provided in the axial direction D1 between compressed stripe portions (high-density regions R1) that are produced when the artificial blood vessel base material VEB is compressed in the axial direction D1 (see FIG. 8). It should be noted that specific numerical values of density of the nodes 1 and the fibrils 2 in the low-density region R2 are not limited as long as they are relatively lower compared to density of the nodes 1 and the fibrils 2 in the high-density region R1.

As shown in FIGS. 2 and 3, in the artificial blood vessel VE of the present embodiment, the high-density regions R1 and the low-density regions R2 are alternately provided in the axial direction D1. This improves flexibility of the artificial blood vessel VE. Specifically, the high-density regions R1 where density of the nodes 1 and the fibrils 2 is high (particularly, density of the node 1, which is relatively hard, is high) and the low-density regions R2 where density of the node 1 and the fibril 2 is low (particularly, density of the node 1, which is relatively hard, is low) are alternately formed in the axial direction D1 of the artificial blood vessel VE, so that the artificial blood vessel VE functions like bellows, improving flexibility of the artificial blood vessel VE.

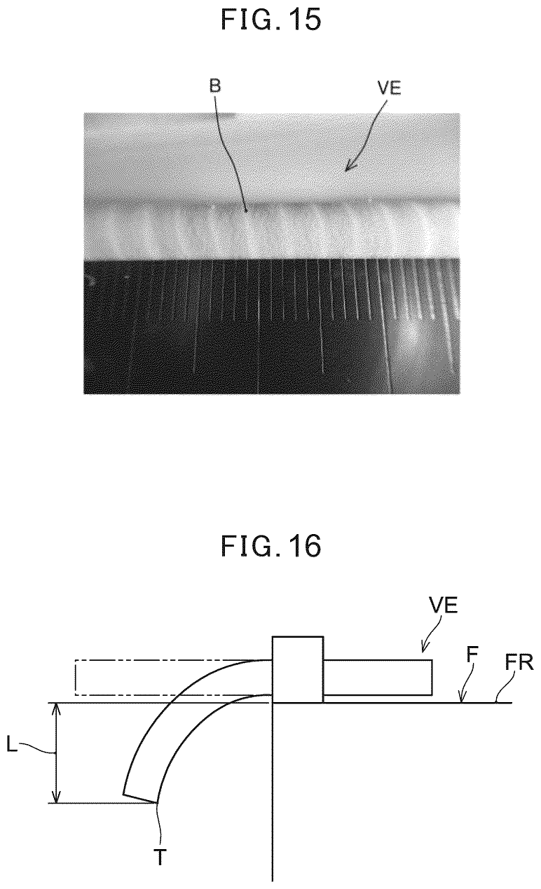

In the present embodiment, as shown in FIGS. 1 and 15, the artificial blood vessel VE further comprises a belt-shaped portion B that extends continuously in a belt shape along the axial direction D1 of the artificial blood vessel VE so as to provide resistance to the artificial blood vessel VE (artificial blood vessel base material VEB) extending to a predetermined length or more in the axial direction D1 after being compressed in the axial direction D1.

The belt-shaped portion B, which will be described in detail below, is a site that provides resistance to the extension of the artificial blood vessel base material VEB so that the artificial blood vessel base material VEB does not extend beyond the predetermined length or more when the artificial blood vessel base material VEB extends after being compressed in the axial direction D1 and subsequently released from the compressive force. Here, the “predetermined length” is a length shorter than a length of an artificial blood vessel base material VEB in its natural state before it is compressed, specifically, a length shorter than a length of an artificial blood vessel base material VEB having a similar structure except that it does not have any belt-shaped portion B when it is extended after being compressed, released from the compressive force, and then a sufficient amount of time has passed. More specifically, the “predetermined length” is preferably 60 to 80%, and further preferably 65 to 75%, of the length of the artificial blood vessel in its natural state before the artificial blood vessel base material VEB is compressed.

A structure and a method of forming a belt-shaped portion B are not particularly limited as long as they are configured to provide resistance to extension of the compressed artificial blood vessel VE (artificial blood vessel base material VEB). For example, the belt-shaped portion B is made harder compared to the remaining sites where the belt-shaped portion B is not formed (sites where the high-density regions R1 and the low-density regions R2 are alternately formed). As a result, even if the remaining sites, which are softer and relatively more easily extend compared to the belt-shaped portion B, attempt to extend in the axial direction D1, the belt-shaped portion B provides resistance to the extension of the remaining sites. The belt-shaped portion B may be formed, for example, by being locally hardened at a predetermined position of the artificial blood vessel base material VEB through heat treatment or the like (e.g., laser baking, heating with a heater, etc.), by attaching tape to the artificial blood vessel base material VEB in a predetermined pattern, or by locally applying a pressing force in a predetermined pattern.

The belt-shaped portion B extends continuously in a belt shape along the axial direction D1 of the artificial blood vessel VE. Here, “extending continuously along the axial direction D1” means that the belt-shaped portion B is connected from one side to the other side in the axial direction D1 so as to provide resistance to the artificial blood vessel base material VEB extending to the predetermined length or more. In the present embodiment, the belt-shaped portion B extends continuously in the axial direction D1 while being inclined with respect to the axial direction D1, but a part of the belt-shaped portion B may have a portion that is parallel to the axial direction D1.

A shape of the belt-shaped portion B is not particularly limited as long as it extends continuously along the axial direction D1 so as to provide resistance to extension of the compressed artificial blood vessel VE (artificial blood vessel base material VEB) as mentioned above. In the present embodiment, as shown in FIGS. 1 and 2, the belt-shaped portion B is provided so that a region where the high-density regions R1 and the low-density regions R2 are alternately formed is arranged between a part P1 of the belt-shaped portion B at one point in the axial direction D1 of the artificial blood vessel VE (see FIG. 1) and the other part P2 spaced apart from the part P1 in the axial direction D1 (see FIG. 1).

In the present embodiment, the belt-shaped portion B extends in a spiral shape around the axis of the artificial blood vessel VE, as shown in FIG. 1. In this case, resistance of the artificial blood vessel VE to an outward force in the radial direction D3 is enhanced. Moreover, as the belt-shaped portion B extends in the spiral shape, the belt-shaped portion B functions like a coil spring, improving a shape retention property of the artificial blood vessel VE and making it easier to suppress the artificial blood vessel VE from extending to the predetermined length or more. Instead of the spiral shape mentioned above, the belt-shaped portion may comprise, for example, a plurality of ring-shaped portions spaced apart in the axial direction D1 and an axial portion connecting the ring-shaped portions to each other in the axial direction D1.

In the present embodiment, with the above-mentioned belt-shaped portion B provided, the site where the high-density region R1 and the low-density region R2 are alternately arranged, which is arranged adjacent to the belt-shaped portion B, is suppressed from extending. Accordingly, a part that functions like bellows, which is formed by the high-density regions R1 and low-density regions R2 alternately arranged in the axial direction D1, is suppressed from overextending to deteriorate in flexibility. Moreover, when the artificial blood vessel VE is in an unloaded state where no force is applied thereto (in other words, when it is in a free length state with no residual stress), the belt-shaped portion B provides resistance to extension of the high-density regions R1 and low-density regions R2, thereby restricting extension of the artificial blood vessel VE. On the other hand, the resistance force of the belt-shaped portion B is designed to allow the extension of the high-density regions R1 and low-density regions R2 when an external force is applied to the artificial blood vessel VE. Thus, the high-density regions R1 and the low-density regions R2 are maintained in a state where they do not overextend in the axial direction D1 by the belt-shaped portion B but also easily extend and contract in the axial direction D1. As shown by the two-dot chain lines in FIG. 1, when a bending force is applied to the artificial blood vessel VE, the high-density regions R1 and the low-density regions R2 easily contract in an inner part P3 of a curved portion of the artificial blood vessel VE and easily extend in an outer part P4 of the curved portion of the artificial blood vessel VE. Thus, the artificial blood vessel VE becomes easy to bend. Accordingly, as the high-density regions R1 and the low-density regions R2 are maintained in the state where they do not overextend in the axial direction D1 by the belt-shaped portion B but also easily extend and contract in the axial direction D1, a change in length of the artificial blood vessel VE over time (shortening or extending of the length of the artificial blood vessel VE relative to the designed length due to the artificial blood vessel VE being left for a predetermined period of time) can be suppressed.

An angle θ of the spiral belt-shaped portion B with respect to an axial line X of the artificial blood vessel VE (see FIG. 1) is not particularly limited, but it is, for example, in a finished state of the artificial blood vessel VE, preferably greater than 45°, more preferably 50 to 80°, and further preferably 60 to 70°. When the angle θ of the spiral belt-shaped portion B with respect to the axial line X is within the above-described ranges, the artificial blood vessel VE becomes easy to compress and extend, so that flexibility of the artificial blood vessel VE can be improved. The belt-shaped portion B may be formed by heat-treating the artificial blood vessel base material VEB in a compressed state, as mentioned below, and in this case, the above-described angle θ is an angle in a state where the compression of the artificial blood vessel base material VEB is released. In addition, when the belt-shaped portion B is formed during compression of the artificial blood vessel base material VEB, an angle for heat treatment when forming the belt-shaped portion B may be set taking into consideration a compression rate of the artificial blood vessel base material VEB and an extension rate when the artificial blood vessel base material VEB extends from the compressed state, etc.

A width (a length in the axial direction D1) of the belt-shaped portion B is not particularly limited as long as the belt-shaped portion B is configured to provide resistance to extension of the compressed artificial blood vessel VE (artificial blood vessel base material VEB). The width of the belt-shaped portion B can be appropriately changed depending on performances, such as flexibility, required for the artificial blood vessel VE. The width of the belt-shaped portion B is not limited, but can be, for example, ⅙ to ¼ of a width of a portion other than the belt-shaped portion B (a portion where the high-density regions R1 and the low-density regions R2 are alternately arranged), in the artificial blood vessel VE In other words, a width of a portion sandwiched between the belt-shaped portions B in the axial direction D1 is 4 to 6 times the width of belt-shaped portion B.

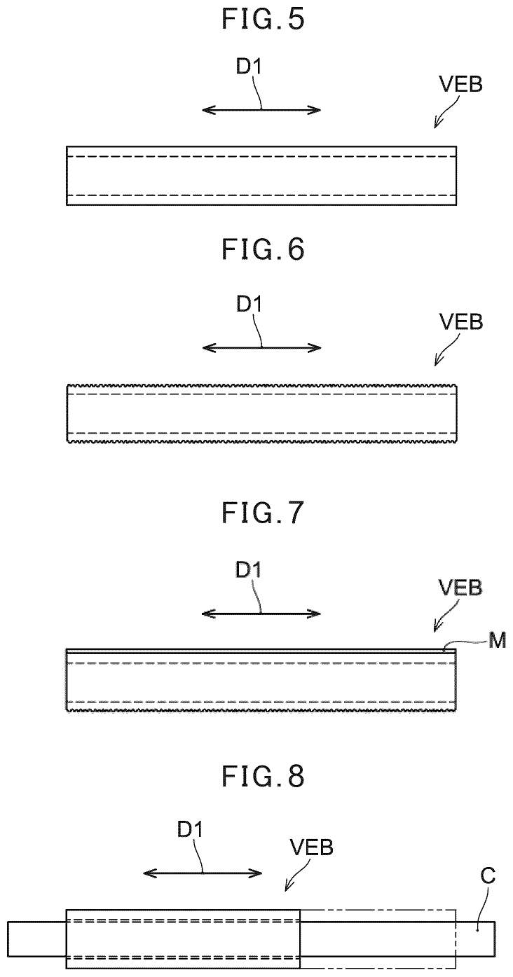

Next, one example of a method of manufacturing an artificial blood vessel VE will be described with reference to the schematic diagrams of FIGS. 5 to 12. It should be noted that the manufacturing method described below is merely one example, and the artificial blood vessel VE of the present invention is not limited to the manufacturing method below, and may be manufactured by another manufacturing method as long as it has features described in claims.

First, as shown in FIG. 5, a tubular artificial blood vessel base material VEB composed of ePTFE having nodes 1 and fibrils 2 formed between the nodes 1 is provided (a preparation step of an artificial blood vessel base material). This artificial blood vessel base material VEB is an ePTFE tube elongated at a predetermined elongating ratio. The artificial blood vessel base material VEB can be obtained, for example, by mixing an unsintered PTFE powder with a lubricant to prepare a mixture, extruding the mixture into a tube using a ram extruder, and then elongating the tube in an axial direction D1 at a desired elongating ratio.

Next, the entire surface of the artificial blood vessel base material VEB is baked (a first baking step, see FIG. 6). Specifically, the entire surface of the artificial blood vessel base material VEB is heated and baked. This baking makes the entire surface of the artificial blood vessel base material VEB rough. As a result, a marker M, which will be mentioned later, becomes easily settled on the surface of the artificial blood vessel base material VEB when the marker Mis applied thereto.

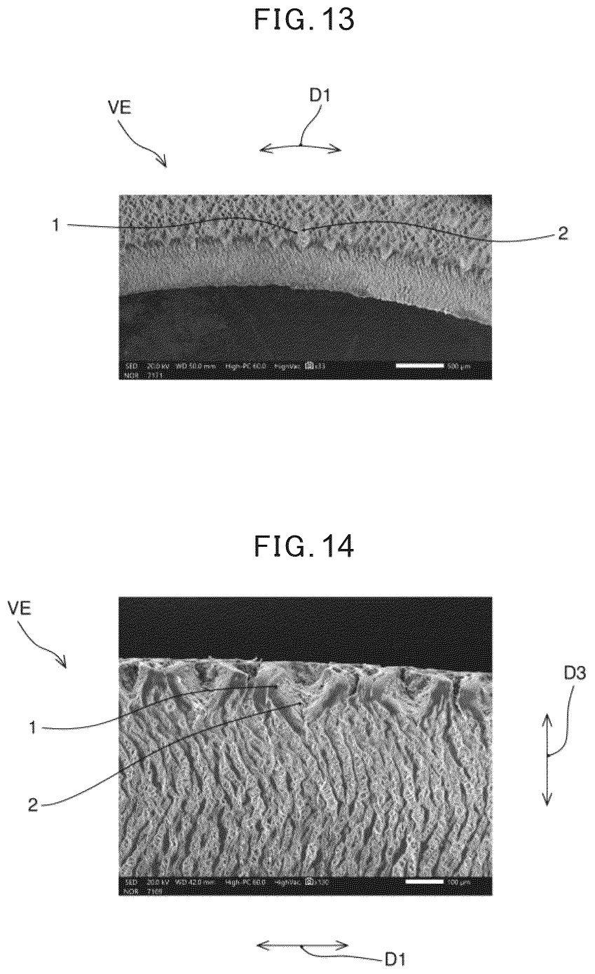

After the entire surface of the artificial blood vessel base material VEB is baked, a linear marker M extending in the axial direction D1 is applied onto the surface of the artificial blood vessel base material VEB (a marker application step, see FIG. 7). The marker M is provided to check linearity of the artificial blood vessel VE. In the present embodiment, the marker M is composed of cobalt and is applied as a single linear coating layer extending in the axial direction D1 of the artificial blood vessel base material VEB. After the marker M is applied, the entire artificial blood vessel base material VEB is further baked (a second baking step, not shown). Specifically, the entire outer periphery of the artificial blood vessel base material VEB is heated, whereby the artificial blood vessel base material VEB is baked. The artificial blood vessel base material VEB is thereby finished. It was confirmed that, as the outer periphery of the artificial blood vessel base material VEB is heated and baked by a heating means in the first baking step, the second baking step, etc., the nodes 1 and fibrils 2 on the surface of the artificial blood vessel base material VEB are partially melted and densely packed so as to come closer to each other, forming concave and convex portions that are repeated along the axial direction D1 (see FIGS. 13 and 14). The concave portions between the nodes 1 thereby become deeper compared to those of the artificial blood vessel base material VEB before baked, and the concaves and convexes of the artificial blood vessel base material VEB are clearly formed. Thus, compared with an artificial blood vessel VE whose surface is flat or having a small difference in height of the concaves and convexes, the artificial blood vessel VE becomes improved in flexibility and easily becomes settled onto surrounding tissues when placed in a living body.

Next, as shown in FIG. 8, the artificial blood vessel base material VEB is compressed in the axial direction D1 of the artificial blood vessel base material VEB in a state where a core member C is inserted inside the artificial blood vessel base material VEB (a first compression step). This step is carried out, for example, by applying a force to the artificial blood vessel base material VEB in the axial direction D1 with the core member inserted inside the artificial blood vessel base material VEB. A compression ratio of the artificial blood vessel base material VEB (a percentage of a length of the artificial blood vessel base material VEB after compressed (the state indicated by the solid line in FIG. 8) to the original state of the artificial blood vessel base material VEB (the state indicated by the two-dot chain line in FIG. 8)) is not particularly limited, but can be, for example, 40 to 70%, preferably 50 to 60%.

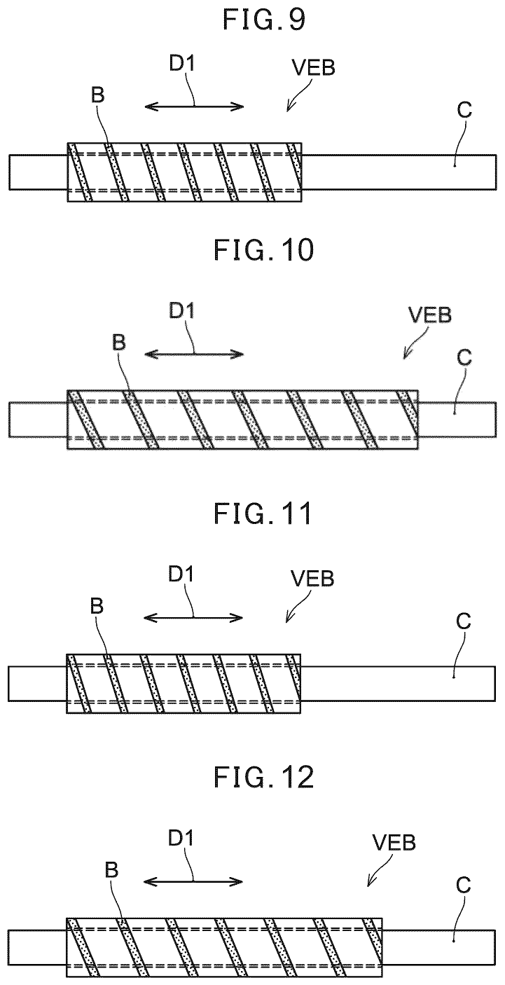

Next, a belt-shaped portion B is provided on the artificial blood vessel base material VEB compressed in the above-mentioned first compression step. The belt-shaped portion B extends continuously in a belt shape along the axial direction D1 of the artificial blood vessel base material VEB so as to provide resistance to the artificial blood vessel VE extending to the predetermined length or more in the axial direction D1 (a belt-shaped portion forming step, see FIG. 9). The method of forming the belt-shaped portion B is not particularly limited. For example, the belt-shaped portion B is formed by baking the surface of the compressed artificial blood vessel base material VEB in the predetermined pattern using the heating means that partially heats the surface. In the present embodiment, the belt-shaped portion B is formed in a spiral shape around the axis of the compressed artificial blood vessel base material VEB. More specifically, the artificial blood vessel base material VEB is heated with the heating means being moved in the axial direction D1 while being rotated around the axis (or heated with the heating means being moved in a spiral shape relative to the artificial blood vessel base material VEB), thereby forming a spiral belt-shaped portion B.

Next, the force compressing the artificial blood vessel base material VEB is released to extend the artificial blood vessel base material VEB to the predetermined length (a first extension step, see FIG. 10). In this first extension step, for example, the artificial blood vessel base material VEB is extended to a length that is 75 to 85% of the length of the artificial blood vessel base material VEB in the above-mentioned preparation step of the artificial blood vessel base material.

After the artificial blood vessel base material VEB is extended in the first extension step, the present embodiment further comprises, in addition to the first compression step and the first extension step, a step of re-compressing the extended artificial blood vessel base material VEB one or more times (an additional compression step) and a step of re-extending the artificial blood vessel base material VEB compressed in the additional compression step (an additional extension step) (see FIGS. 11 and 12). The artificial blood vessel base material VEB is removed from the core member C and extended to a desired length required for the artificial blood vessel VE.

In a case where compression and extension of the artificial blood vessel base material VEB are defined as one set, by performing multiple sets of compression and extension in these additional compression step and additional extension step, compression stripes, in which the high-density regions R1 and the low-density regions R2 are alternately formed, can be formed more sharply compared to the case where the artificial blood vessel base material VEB is compressed and extended only once. Furthermore, in these additional compression step and additional extension step, compression and extension are repeated in the axial direction D1 of the artificial blood vessel base material VEB, causing the hard nodes 1 to bend repeatedly, so that a bending crease (a folding crease) is formed (in a single compression step, a bending crease is not formed or disappears when the artificial blood vessel base material VEB is extended). This allows the hard node 1 portions to gradually become flexible, making the artificial blood vessel VE flexible. In the present embodiment, the above-mentioned additional compression step and additional extension step have a synergistic effect of sharpening the compression stripes in which the high-density regions R1 and the low-density regions R2 are alternately formed and forming a bending crease (a folding crease) of the hard nodes 1, thereby making it possible to increase flexibility of the artificial blood vessel VE.

Moreover, in the present embodiment, the formation of the belt-shaped portion B suppresses the artificial blood vessel VE from extending to the predetermined length or more. Therefore, the above-mentioned compression stripes and bending creases of the nodes 1 become easy to remain, so that the artificial blood vessel VE becomes easily maintained in a flexible state. That is, even if high-density regions R1 and low-density regions R2 appear alternately, density of the high-density regions R1 will gradually decrease due to extension of the artificial blood vessel base material VEB when the artificial blood vessel base material VEB is left for a sufficient period of time. However, the belt-shaped portion B prevents the extension of the artificial blood vessel base material VEB, thereby suppressing the high-density regions R1 from overextending in the axial direction D1. Moreover, the bending crease (folding crease) portions of the nodes 1 are also maintained by the belt-shaped portion B. Thus, flexibility of the artificial blood vessel VE can be improved by maintaining the portions where the high-density regions R1 and the low-density regions R2 are alternately formed and the portions having bending crease of the nodes 1. In addition, since the artificial blood vessel VE is maintained in a state where it easily extends and contracts in the axial direction D1, a tensile limit can be determined by the extending of the artificial blood vessel VE when pulling an anastomosis thread, therefore making the procedure easier.

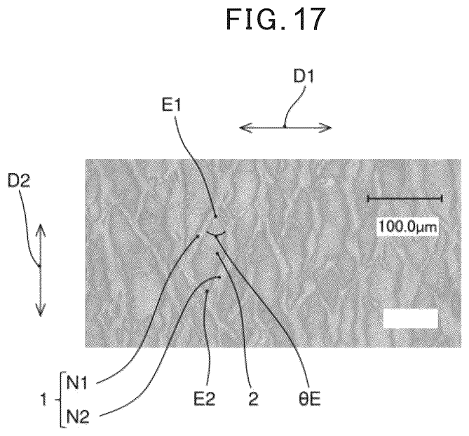

SEM photographs of the surface and the cross section of the artificial blood vessel VE manufactured by the above-mentioned manufacturing method are shown in FIGS. 4, 13, and 14. In the present embodiment, as shown in FIG. 4, on the surface of the artificial blood vessel VE, a portion of the node 1 that extends in a direction perpendicular to an extending direction of the fibril 2 (portion that extends in the vertical direction in FIG. 4) is curved in a wavy manner. In this way, when the additional compression step and the additional elongation step were included, the node 1 was being displaced in a wavy manner in the axial direction D1 as it progressed in the circumferential direction D2. Moreover, as shown in FIGS. 13 and 14, it was observed that, on the surface of the artificial blood vessel VE, depths of and widths in the axial direction D1 of a concave and a convex formed between a pair of nodes 1, 1 became larger. In addition, in the photograph shown in FIG. 17, the nodes 1 of the artificial blood vessel VE include a pair of node portions N1, N2 adjacent to each other in the axial direction D1. The pair of node portions N1, N2 are connected by a pair of contact points E1, E2 on both sides in the circumferential direction D2 of the artificial blood vessel VE. In the present embodiment, the pair of contact points E1, E2 have a folding crease (crease portion) configured so that an angle θE formed by the pair of node portions N1, N2 changes at the pair of contact points E1, E2. Specifically, due to the above-mentioned manufacturing method (the additional compression step and the additional extension step), the contact points E1, E2 have folding creases (creases) and are therefore easy to bend. Therefore, in extending and contracting in the axial direction D1, the angle θE formed between the node portion N1 and the node portion N2 becomes easy to change. Due to the change in angle θE, parts of the node portions N1 and N2 connected by the contact points E1 and E2 become easy to come closer to each other or move away from each other, facilitating extension and contraction of the artificial blood vessel VE.

It should be noted that the number of times the additional compression step and the additional extension step are performed are not particularly limited, but can be, for example, 1 to 20 times, preferably 5 to 15 times. Moreover, a total length of the artificial blood vessel VE in the axial direction D1 after completion of the final additional extension step is not limited, but is preferably 60 to 80%, and more preferably 65 to 75% of the length of the artificial blood vessel base material VEB in the preparation step of the artificial blood vessel base material (in the state shown in FIGS. 5 to 7). Furthermore, the total length of the artificial blood vessel VE in the axial direction D1 after completion of the final additional extension step is shorter than a total length of the artificial blood vessel base material VEB after the first extension step (the state shown in FIG. 10).

Next, an effect of improving flexibility depending on presence or absence of an additional compression step and an additional extension step will be described. Flexibility was evaluated after attaching the artificial blood vessel VE so that a part of 150 mm from the tip thereof is protruded from a fixing base F, as shown in FIG. 16, by measuring a vertical length L from a reference plane FR of the fixing base F to the tip T of the curved artificial blood vessel VE. As samples, artificial blood vessel base materials of the same conditions were used for both those without the additional compression step and the additional extension step (Comparative example) and those with the additional compression step and the additional extension step (Example). Specifically, artificial blood vessel base materials having a wall thickness of 0.6 mm, an outer diameter of 7.2 mm, and a length of 207 mm, and made of ePTFE with an elongating ratio of 2.9 were used. Regarding the manufacturing method, in Example, the additional compression step and the additional extension step were performed ten times, whereas in Comparative example, the additional compression step and the additional extension step were not performed, and the compression step and the extension step were performed only once. As a result, in Comparative example, the vertical length L from the reference plane FR of the fixing base F to the tip T of the curved artificial blood vessel VE was 17.3 mm. In contrast, in Example, the vertical length L from the reference plane FR of the fixing base F to the tip T of the curved artificial blood vessel VE was 80 mm. It was thereby found that flexibility of the artificial blood vessel VE is greatly improved by performing the additional compression step and the additional extension step.

Although the embodiments of the present invention have been described above, the present invention is not limited to the above-described embodiments. The above-described embodiments mainly explain the inventions having the following configurations.
(1) An artificial blood vessel composed of ePTFE having nodes and fibrils formed between the nodes,
(2) wherein high-density regions and low-density regions are alternately provided in an axial direction of the artificial blood vessel, wherein, in the high-density regions, the nodes and the fibrils are in a compressed and densely packed state in the axial direction, and in the low-density regions, the nodes and the fibrils are in a lower density state compared to the high-density region.
(3) The artificial blood vessel of (1), further comprising:
(4) a belt-shaped portion that extends continuously in a belt shape along the axial direction of the artificial blood vessel so as to provide resistance to the artificial blood vessel extending to a predetermined length or more in the axial direction after being compressed in the axial direction.

(3) The artificial blood vessel of (1) or (2), wherein the nodes include a pair of node portions adjacent to each other in the axial direction, wherein the pair of node portions are connected by a pair of contact points on both sides in a circumferential direction of the artificial blood vessel, and wherein the pair of contact points have a folding crease configured so that an angle formed by the pair of node portions changes at the pair of contact points.

(4) A method of manufacturing an artificial blood vessel, comprising the steps of:
a) providing a tubular artificial blood vessel base material composed of ePTFE having nodes and fibrils formed between the nodes;
b) compressing the artificial blood vessel base material in an axial direction of the artificial blood vessel base material in a state where a core member is inserted inside the artificial blood vessel base material;
c) releasing a force compressing the artificial blood vessel base material to extend the artificial blood vessel base material;
d) re-compressing the extended artificial blood vessel base material one or more times; and
e) re-extending the artificial blood vessel base material compressed in the step d).

(5) The method of manufacturing an artificial blood vessel of (4),

further comprising the step of providing a belt-shaped portion on the artificial blood vessel base material compressed in the step b), wherein the belt-shaped portion extends continuously in a belt shape along the axial direction of the artificial blood vessel base material so as to provide resistance to the artificial blood vessel extending to the predetermined length or more in the axial direction.

(6) The method of manufacturing an artificial blood vessel of (4) or (5), wherein the belt-shaped portion extends in a spiral shape around an axis of the artificial blood vessel.

1. An artificial blood vessel composed of ePTFE having nodes and fibrils formed between the nodes,
wherein high-density regions and low-density regions are alternately provided in an axial direction of the artificial blood vessel, wherein, in the high-density regions, the nodes and the fibrils are in a compressed and densely packed state in the axial direction, and in the low-density regions, the nodes and the fibrils are in a lower density state compared to the high-density region.

2. The artificial blood vessel of claim 1, further comprising:
a belt-shaped portion that extends continuously in a belt shape along the axial direction of the artificial blood vessel so as to provide resistance to the artificial blood vessel extending to a predetermined length or more in the axial direction after being compressed in the axial direction.

3. The artificial blood vessel of claim 1, wherein the nodes include a pair of node portions adjacent to each other in the axial direction, wherein the pair of node portions are connected by a pair of contact points on both sides in a circumferential direction of the artificial blood vessel, and wherein the pair of contact points have a folding crease configured so that an angle formed by the pair of node portions changes at the pair of contact points.

4. A method of manufacturing an artificial blood vessel, comprising the steps of:
a) providing a tubular artificial blood vessel base material composed of ePTFE having nodes and fibrils formed between the nodes;
b) compressing the artificial blood vessel base material in an axial direction of the artificial blood vessel base material in a state where a core member is inserted inside the artificial blood vessel base material;
c) releasing a force compressing the artificial blood vessel base material to extend the artificial blood vessel base material;
d) re-compressing the extended artificial blood vessel base material one or more times; and
e) re-extending the artificial blood vessel base material compressed in the step d).

5. The method of manufacturing an artificial blood vessel of claim 4, further comprising the step of providing a belt-shaped portion on the artificial blood vessel base material compressed in the step b), wherein the belt-shaped portion extends continuously in a belt shape along the axial direction of the artificial blood vessel base material so as to provide resistance to the artificial blood vessel extending to the predetermined length or more in the axial direction.

6. The method of manufacturing an artificial blood vessel of claim 5, wherein the belt-shaped portion extends in a spiral shape around an axis of the artificial blood vessel.
